(19) United States
(12) Patent Application Publication
(10) Pub. No.: US 2019/0211291 A1
(43) Pub. Date: Jul. 11, 2019

(54) PRODUCING RECOVERABLE OIL FROM FERMENTATION PROCESSES

(71) Applicants: DIREVO Industrial Biotechnology GmbH, Cologne (DE); BASF Enzymes, LLC, San Diego, CA (US)

(72) Inventors: Vitaly Svetlichny, Cologne (DE); Marco Kraemer, Pulheim (DE); Klaudija Milos, Cologne (DE)

(21) Appl. No.: 16/357,903

(22) Filed: Mar. 19, 2019

Related U.S. Application Data
(63) Continuation-in-part of application No. 15/501,533, filed on Feb. 3, 2017.
Application No. 15/501,533 is a continuation-in-part of application No. 14/767,148, filed on Aug. 11, 2015.
Application No. 14/767,148 is a continuation-in-part of application No. 14/627,753, filed on Feb. 20, 2015.
Application No. 14/627,753 is a continuation of application No. 13/995,079, filed on Aug. 19, 2013, now Pat. No. 8,962,286.
(60) Provisional application No. 62/033,349, filed on Aug. 5, 2014.
(60) Provisional application No. 61/425,893, filed on Dec. 22, 2010.

(30) Foreign Application Priority Data
Feb. 21, 2013 (EP) 13156260.5
Aug. 5, 2014 (EP) 14179841.3

(51) Int. Cl. C12F 3/10; A23K 50/30; C12P 7/06; C12P 7/14; C12N 9/24; C12N 9/14; A23K 50/75; A23K 10/38; C12C 7/053; C12N 9/42

(52) U.S. Cl. CPC C12F 3/10; Y02P 60/873; C12P 7/06; C12P 7/14; C12N 9/2491; C12N 9/14; A23K 50/75; A23K 10/38; C12C 7/053; C12N 9/2402; C12N 9/2437; C12N 9/244; C12N 9/2482; Y02E 50/17; C12Y 302/01004; C12Y 302/01015; A23K 50/30

(57) ABSTRACT

A method of recovering oil, which includes (a) converting a starch-containing material into dextrins with an alpha-amylase; (b) saccharifying the dextrins using a carbohydrate source generating enzyme to form a sugar; (c) fermenting the sugar in a fermentation medium into a fermentation product using a fermenting organism, wherein the fermentation medium comprises a xylanase and a pectinase; (d) distilling the fermentation product to form a whole stillage; (e) separating the whole stillage into thin stillage and wet cake; and (f) recovering the oil from the thin stillage.

### CROSS REFERENCE TO RELATED APPLICATIONS

This is a continuation in part of U.S. patent application Ser. No. 15/501,533, which is a US national phase application of PCT/EP2015/064090, filed Jun. 23, 2015, which claims priority to U.S. provisional patent application No. 62/033,349, filed Aug. 5, 2014, now expired, and European patent application no. 14179841.3, filed Aug. 5, 2014. Each application cited in this paragraph is herein incorporated by reference in its entirety.

This is also a continuation in part of U.S. patent application Ser. No. 14/767,148, which is a US national phase application of PCT/EP2013/055918, filed Mar. 21, 2013, which claims priority to European patent application no. 13156260.5, filed Feb. 21, 2013. Each application cited in this paragraph is herein incorporated by reference in its entirety.

This is also a continuation in part of U.S. patent application Ser. No. 14/627,753, which is a continuation of U.S. patent application Ser. No. 13/995,079, now U.S. Pat. No. 8,962,286, which is a US national phase application of PCT/EP2011/006473 filed Dec. 21, 2011, which claims benefit of priority to U.S. provisional patent application Ser. No. 61/425,893 filed Dec. 22, 2010, now expired. Each application cited in this paragraph is herein incorporated by reference in its entirety.

### FIELD OF THE INVENTION

The present disclosure relates to an improved process of producing recoverable oil from fermentation processes.

### BACKGROUND OF THE INVENTION

Fermentation products, such as ethanol, are produced by first degrading starch-containing material into fermentable sugars by liquefaction and saccharification and then converting the sugars directly or indirectly into the desired fermentation product using a fermenting organism. Liquid fermentation products such as ethanol are recovered from the fermented mash (often referred to as “beer” or “beer mash”), e.g., by distillation, which separate the desired fermentation product from other liquids and/or solids. The remaining faction, referred to as “whole stillage”, is dewatered and separated into a solid and a liquid phase, e.g., by centrifugation. The solid phase is referred to as “wet cake” (or “wet grains” or “WDG”) and the liquid phase (supernatant) is referred to as “thin stillage”. Dewatered wet cake is dried to provide “Distillers Dried Grains” (DDG) used as nutrient in animal feed. Thin stillage is typically evaporated to provide condensate and syrup (or “thick stillage”) or may alternatively be recycled directly to the slurry tank as “backset”. Condensate may either be forwarded to a methanator before being discharged or may be recycled to the slurry tank. The syrup consisting mainly of limit dextrins and non-fermentable sugars may be blended into DDG or added to the wet cake before drying to produce DDGS (Distillers Dried Grain with Solubles).

Ethanol plants have struggled to maintain profitability, which is highly variable depending upon corn price, demand and price of DDGS, tax credits, gasoline consumption, ethanol exports, and changes to the Renewable Fuels Standard (RFS) mandates. New technologies for energy savings, higher yield of ethanol and higher value for co-products as well as various oil separation technologies contribute to the profitability of producing ethanol.

Corn oil recovery has been recognized as one of the keys in keeping many ethanol plants profitable in times of tight margins by improving operating income and diversifying plant revenue streams. During the past two years, the U.S. ethanol industry has widely implemented advanced corn oil extraction technology. It was estimated that at the end of 2013 about 80% of U.S. ethanol plants were recovering corn oil. According to a report from the U.S. Energy Information Administration⁴, although ethanol facilities with corn oil extraction have slightly higher production costs, their profit margins due to corn oil sales have remained positive and higher than for plants without oil recovery. Due to negative margins, many ethanol plants without oil recovery have chosen to shutdown.

Corn oil recovered as a co-product of ethanol production, also referred to as distiller's corn oil, is an economically attractive and renewable feedstock for biodiesel production. Oil removal from DDGS may also benefit handling and transport of DDGS (less caking and improved flow properties) and expand the use of low-fat DDGS in non-ruminant livestock.

Furthermore, the use of the distillers corn oil, which is inedible feedstock for biodiesel production, would not impact the cost and availability of oil for food.

Reviews of current technologies for corn oil recovery from dry-grind ethanol plants were given in previous reports. Physical pretreatment on dry corn, front- and back-end oil recovery in combination with heating, use of demulsifiers and polar solvents have significantly improved corn oil recovery compared to several years ago. Considerable efforts have been made to use enzymes to facilitate oil separation.

U.S. Pat. No. 6,433,146 discloses extracting oil and zein from corn or corn processing by-products using ethanol. U.S. Pat. No. 7,601,858 discloses a method for recovering oil from a concentrated byproduct, such as thin stillage formed during a dry milling process used for producing ethanol. The method includes forming a concentrate from the byproduct, e.g., by evaporating the by-product, and recovering oil from the concentrate. U.S. Pat. No. 7,608,729 discloses a method of freeing the bound oil present in whole stillage and thin stillage by heating the stillage to a temperature sufficient to at least partially separate, or bind, the oil from the stillage. U.S. Application Publication No. 2010/0058649 discloses a method of separating an oil fraction from a fermentation product, adjusting the pH of the oil fraction, and recovering the oil from the oil fraction.

It is an object of the present disclosure to provide improved methods for increasing the amount of recoverable oil from fermentation processes.

### SUMMARY OF THE INVENTION

The present invention relates to processes of fermenting a starch-containing material into a fermentation product comprising a fermentation step in the presence of a xylanase in combination with a pectinase on oil partitioning during post-fermentation processing. In particular, the enzyme(s) were added during simultaneous saccharification and fermentation. The finished beer was subjected to beer well incubation, distillation, and then decanting to separate thin stillage from the solids.

In one aspect, the present disclosure pertains to a process of producing recoverable oil from fermentation processes, wherein liquefied whole grain mash is thinned by treatment with an efficient amount of enzyme activity of a xylanase in combination with a pectinase.

In a further aspect, the present disclosure pertains to a process for producing recoverable oil from fermentation processes, wherein the process sequentially comprises the following steps: a) milling whole grain; b) liquefying the gelatinised milled whole grain, in the presence of an alpha-amylase; c) saccharifying the liquefied material in the presence of a glucoamylase; d) fermentation with a micro-organism; e) distillation of fermented and saccharified material, providing an ethanol fraction, wherein the liquefied mash is thinned by subjection to an effective amount enzyme activity of a xylanase and a pectinase.

The present disclosure relates further to methods for the improvement of the quality of the by-products or residues derived from fermented mash comprising the steps of: i) subjecting the fermented mash during or after the fermentation to an enzyme composition comprising an enzyme or a mixture of enzymes capable of degrading one or more fermented mash components, ii) separating the desired fermentation product.

The present disclosure also relates to methods of producing ethanol from starch containing material, said method comprising the steps of:
i) Converting starch containing material to fermentable sugars;
ii) Fermentation of the fermentable sugars with a microorganism to fermented mash;
iii) Subjecting the fermented mash after the fermentation process to an enzyme composition comprising an enzyme or a mixture of enzymes; and
iv) Separation of the ethanol in the fermented mash by distillation.

The present disclosure also relates to uses of an enzyme composition comprising a beta-1,3-glucanase and/or a xylanase for the improvement of the nutritional quality of the by-products or residues derived from fermented mash in a fermentative production process.

The present disclosure also relates to methods for the manufacturing of an enzymes composition used for treating fermented mash of in an fermentative production process to improve the nutritional quality of a by-product or residue and/or the process ability of the production process, comprising:
a) inoculating the by-product or residue with at least one filamentous fungus;
b) fermenting the by-product or residue; and
c) separating at least one enzyme from the fermented by-product or residue.

The present disclosure also relates to methods of producing a feed co-product, comprising:
a) converting a starch-containing material into dextrins with an alpha-amylase;
b) saccharifying the dextrins using a carbohydrate source generating enzyme to form a sugar;
c) fermenting the sugar in a fermentation medium into a fermentation product using a fermenting microorganism;
d) adding after the fermenting process an enzyme composition comprising at least a xylanase and/or a beta 1,3-glucanase to the fermented mash;
e) distilling the fermentation product to form a whole stillage;
f) separating the whole stillage into thin stillage and wet cake;
g) de-oiling the thin stillage to form soluble with less oil; and
h) recovering the feed co-product from the wet cake and soluble with less oil.

The present disclosure relates to a process of fermenting a starch-containing material into a fermentation product comprising a fermentation step without the presence of a beta 1,3-glucanase and/or a xylanase. After the fermentation an enzyme composition is added to the fermented mash for an improvement of the by-products like the fibrous by-products such as spent brewer's grains, dried distiller's grains, dried distiller's soluble, distiller's dried grains with soluble, wet grains, and mixtures thereof.

The present disclosure relates further to a method for manufacturing an enzyme composition used for treating fermented mash in a fermentation production process to improve the nutritional quality of a by-product or residue and/or the processability of the production process, the method comprising:
a) inoculating whole stillage or distiller's wet grain (DWG) with at least one filamentous fungus;
b) fermenting the inoculated whole stillage or DWG;
c) separating a supernatant comprising at least two enzymes from the fermented whole stillage or DWG for the treatment of fermented mash, wherein the at least two enzymes are beta-1,3-glucanase and xylanase.

In some embodiments, the filamentous fungus is selected from the group consisting of Rhizopus, Aspergillus, Trichoderma, and a combination thereof.

In some embodiments, the method further comprising separating a mannanase from the whole stillage or DWG.

In some embodiments, the method further comprising separating a pectinase from the whole stillage or DWG.

The present disclosure relates further to a process of producing a prebiotic animal feed product comprising a by-product derived from a fermentative production process comprising the steps of: i) subjecting the fermented mash after the fermentation to an enzyme composition capable of degrading one or more fermented mash components, ii) separating the desired primary fermentation product, iii) separating the fermentation by-product having Unproved prebiotic quality.

The present disclosure relates further to a process of producing a prebiotic animal feed product, the process comprising the steps of:
i) subjecting fermented mash after fermentation to an enzyme composition capable of degrading one or more fermented mash components,
ii) separating a desired primary fermentation product,
iii) separating a fermentation by-product suspected of having improved prebiotic quality,
iv) assaying levels of beta-glucans and manno-oligosaccharides in the separated fermentation by-product, and
v) designating the separated fermentation by-product as a prebiotic for use with animal feed if the separated fermentation by-product has an improved prebiotic quality characterized by increased levels of beta-glucans and manno-oligosaccharides compared to fermented mash.

The present disclosure relates further to a method of producing a prebiotic animal feed product comprising a fermentation by-product having a high content of prebiotics like beta glucans and/or mannan-oligo-saccharides from starch-containing material in an alcohol fermentation production process, said method comprising the steps of:
i) Convening the starch-containing material to fermentable sugars,
ii) Fermentation of the fermentable sugars with a fermenting microorganism to fermented mash,
iii) Separation of the alcohol in the fermented mash by distillation,
iv) Subjecting the remaining whole stillage to an enzyme composition capable of degrading one or more components of the whole stillage to beta glucans and/or mannan-oligo-saccharides,
v) Separation of the by-product.

The present disclosure relates further to a method of producing a prebiotic animal feed product having a high content of beta-glucans and manno-oligosaccharides as prebiotics, the method comprising the steps of:
i) converting starch-containing material to fermentable sugars,
ii) fermenting the fermentable sugars with a fermenting microorganism to fermented mash,
iii) separating alcohol from the fermented mash by distillation,
iv) subjecting remaining whole stillage to an enzyme composition capable of degrading one or more components of the whole stillage to beta glucans and manno-oligosaccharides,
v) assaying levels of beta-glucans and manno-oligosaccharides in the whole stillage by-product, and if enriched for beta-glucans and manno-oligosaccharides,
vi) separating the by-product and designating the by-product as a prebiotic for use with animal feed.

### BRIEF DESCRIPTION OF THE DRAWINGS

FIG. 1 schematically shows an ethanol production process.

FIG. 2 schematically shows an ethanol process including on site fermentation tank for enzyme production based on WDG.

FIG. 3 schematically shows an ethanol process including on site fermentation tank for enzyme production based on whole stillage.

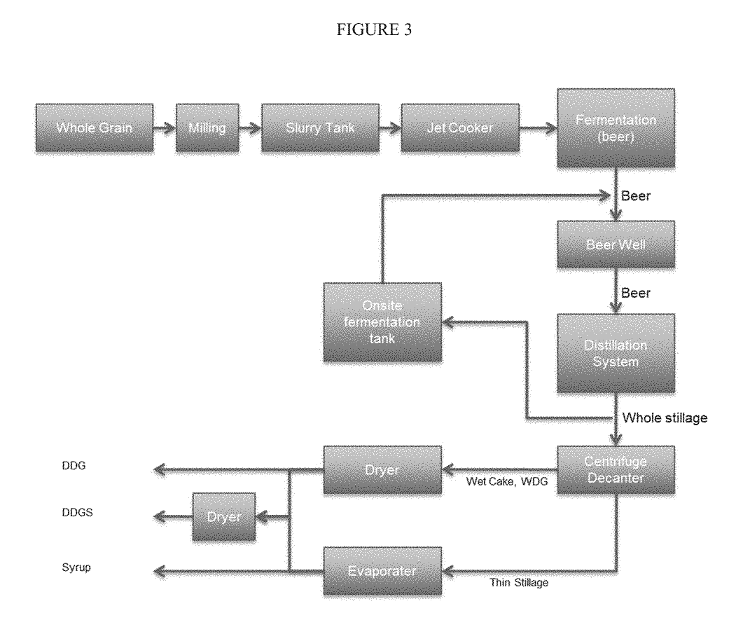

FIG. 4 is a diagram showing the reduction of ADF and NDF in DDGS by using 1,3-β-glucanase.

FIG. 5 is a diagram showing the reduction of ADF and NDF in DDGS by using xylanase.

FIG. 6 is a diagram showing the reduction of ADF and NDF in DDGS by using an enzyme composition comprising 1,3,-β-glucanase and xylanase.

FIG. 7 is a diagram showing the reduction of ADF and NDF in DDGS by using an enzyme composition comprising 1,3,-β-glucanase and xylanase and a pectinase or a protease.

FIG. 8 showing a picture of thin stillage from not enzyme treated beer, whereby no oil separation can be shown.

FIG. 9 showing a picture of thin stillage from enzyme treated beer, whereby a clear separation of the oil forming in thick oil layer is shown.

FIG. 10 is a diagram showing the weight gain of quails fed with different feed stuff.

FIG. 11 is a diagram showing the dewatering capabilities of an enzyme based process according to the present disclosure by using an enzyme composition comprising 1,3,-β-glucanase and xylanase.

FIG. 12 is a diagram showing the improved protein release in a pepsin/HCL solution when using an enzyme composition comprising 1,3,-β-glucanase and xylanase in a process according to the present disclosure.

FIG. 13 is a diagram showing the improved release of free amino groups of DDGS in water when using an enzyme composition comprising 1,3,-β-glucanase and xylanase in a process according to the present disclosure.

FIG. 14 is a diagram showing the increase of beta-glucans in DDGS extract by using beta-glucanase.

FIG. 15 is a diagram showing the increase of mannan-oligo-saccharides (mos) in DDGS extract by using beta-glucanase.

### DESCRIPTION OF THE INVENTION

The object of the present invention is to provide improved methods of increasing the amount of oil recovered from a process for producing a fermentation product.

The addition of a xylanase in combination with a pectinase enhances the oil extraction from thin stillage or syrup following fermentation, which can be used in biodiesel or other biorenewable product production.

In an embodiment, the present invention relates to a process for producing recoverable oil from fermentation processes, wherein the process comprises the steps of:
(a) liquefying a starch-containing material in the presence of an alpha-amylase;
(b) saccharifying the liquefied material obtained in step (a); and
(c) fermenting using a fermenting organism in the presence of a xylanase and a pectinase,
(d) distilling the fermentation product to form a whole stillage;
(e) separating the whole stillage into thin stillage and wet cake; and
(f) recovering the oil from the thin stillage.

In a further embodiment, the present invention relates to a process of recovering oil, comprising
(a) converting a starch-containing material into dextrins with an alpha-amylase;
(b) saccharifying the dextrins using a carbohydrate source generating enzyme to form a sugar;
(c) fermenting the sugar in a fermentation medium into a fermentation product using a fermenting organism, wherein the fermentation medium comprises a xylanase and a pectinase;
(d) distilling the fermentation product to form a whole stillage;
(e) separating the whole stillage into thin stillage and wet cake; and
(f) recovering the oil from the thin stillage.

Stillage is the product which remains after the mash has been converted to sugar, fermented and distilled into ethanol. Stillage can be separated into two fractions, such as, by centrifugation or screening: (1) wet cake (solid phase) and (2) the thin saline (supernatant). The solid fraction or distillers' wet grain (DWG) can be pressed to remove excess moisture and then dried to produce distillers' dried grains (DDG). After ethanol has been removed from the liquid fraction, the remaining liquid can be evaporated to concentrate the soluble material into condensed distillers' solubles (DS) or dried and ground to create distillers' dried solubles (DDS). DDS is often mixed with DDG to form distillers' dried grain with solubles (DDGS). DDG, DDGS, and DWG are collectively referred to as distillers' grain(s).

In one embodiment of the present disclosure enzymes were added during and/or after the fermentation in the production process to the fermented mash and/or the fermentation medium and before the separation step like distillation, where the desired fermentation main product is separated from the rest of the fermented mash. The enzymes according to the present disclosure were capable of degrading components in the fermented mash (beer or beer mash) and/or the fermentation medium.

The phrase “fermentation media” or “fermentation medium” refers to the environment in which fermentation is carried out and comprises the fermentation substrate, that is, the carbohydrate source that is metabolized by the fermenting organism(s).

The fermentation medium may comprise other nutrients and growth stimulator(s) for the fermenting organism(s). Nutrient and growth stimulators are widely used in the art of fermentation and include nitrogen sources, such as ammonia; vitamins and minerals, or combinations thereof. Recovery Subsequent to fermentation, the fermentation product may be separated from the fermentation medium. The fermentation medium may be distilled to extract the desired fermentation product or the desired fermentation product may be extracted from the fermentation medium by micro or membrane filtration techniques. Alternatively, the fermentation product may be recovered by stripping. Methods for recovery are well known in the art.

Surprisingly, the amount of recoverable oil is increased. DDGS following an ethanol production process from corn typically contains about 13% oil, 31% protein and 56% carbohydrates and other components. Removal of some of the oil from the DDGS will improve the quality of the DDGS for the feed market as many feed producers prefer less oil and fat in the DDGS to make high quality feed.

The starch-containing material may be obtained from cereals. Suitable starch-containing material includes corn (maize), wheat, barley, cassava, sorghum, rye, triticale, potato, or any combination thereof.

Corn is the preferred feedstock, especially when the fermentation product is ethanol. The starch-containing material may also consist of or comprise, e.g., a side stream from starch processing, e.g., C6 carbohydrate containing process streams that may not be suited for production of syrups. Beer components include fiber, hull, germ, oil and protein components from the starch-containing feedstock as well as non-fermented starch, yeasts, yeast cell walls and residuals. Production of a fermentation product is typically divided into the following main process stages: a) Reducing the particle size of starch-containing material, e.g., by dry or wet milling; b) Cooking the starch-containing material in aqueous slurry to gelatinize the starch, c) Liquefying the gelatinized starch-containing material in order to break down the starch (by hydrolysis) into maltodextrins (dextrins); d) Saccharifying the maltodextrins (dextrins) to produce low molecular sugars (e.g., DP1-2) that can be metabolized by a fermenting organism; e) Fermenting the saccharified material using a suitable fermenting organism directly or indirectly converting low molecular sugars into the desired fermentation product; f) Recovering the fermentation product, e.g., by distillation in order to separate the fermentation product from the fermentation mash.

As mentioned above beer (or fermented mash) is the fermentation product consisting of ethanol, other liquids and solids of a desired fermentation product. According to the invention the fermentation product may be any fermentation product, including alcohols (e.g., ethanol, methanol, butanol, 1,3-propanediol); organic acids (e.g., citric acid, acetic acid, itaconic acid, lactic acid, gluconic acid, gluconate, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B12, beta-carotene); and hormones. Fermentation is also commonly used in the production of consumable alcohol (e.g., spirits, beer and wine), dairy (e.g., in the production of yogurt and cheese), leather, and tobacco industries. In a preferred embodiment the fermentation product is a liquid, preferably an alcohol, especially ethanol. The beer contemplated according to the invention may be the product resulting from a fermentation product production process including above mentioned steps a) to f). However, the beer may also be the product resulting from other fermentation product production processes based on starch- and/or lignocellulose containing starting material.

The fermenting organism may be a fungal organism, such as yeast, or bacteria. Suitable bacteria may e.g. be Zymomonas species, such as Zymomonas mobiles and E. coli. Examples of filamentous fungi include strains of Penicillium species. Preferred organisms for ethanol production are yeasts, such as e.g. Pichia or Saccharomyces. Preferred yeasts according to the disclosure are Saccharomyces species, in particular Saccharomyces cerevisiae or baker's yeast.

Furthermore, the use of the enzyme compositions according to the present disclosure in the beer mash after the fermentation and before the distillation process can reduce the viscosity of the beer mash through the degradation of fibers and/or the fermentative microorganisms in the beer. The reduction of the fibers in the beer results in a reduction of the fiber content in the by-products. The early degradation of the NSP's has a direct influence on the separation and the drying conditions of the by-products like DDGS in the production process. The lower viscosity results in lower drying temperatures and also in a shorter drying time resulting in an improved quality of the by-products. For example, the temperature sensitive products like proteins and amino acids arc not destroyed.

Further, by adding the enzymes according to the present disclosure to the fermented mash before, the distillation step is an advantage since the enzymes in the enzyme compositions are are inactivated during the distillation.

Processes for producing fermentation products, such as ethanol, from a starch or lignocellulose containing material are well known in the art. The preparation of the starch-containing material such as corn for utilization in such fermentation processes typically begins with grinding the corn in a dry-grind or wet-milling process. Wet-milling processes involve fractionating the corn into different components where only the starch fraction enters into the fermentation process. Dry-grind processes involve grinding the corn kernels into meal and mixing the meal with water and enzymes. Generally two different kinds of dry-grind processes are used. The most commonly used process, often referred to as a “conventional process,” includes grinding the starch-containing material and then liquefying gelatinized starch at a high temperature using typically a bacterial alpha-amylase, followed by simultaneous saccharification and fermentation (SSF) carried out in the presence of a glucoamylase and a fermentation organism. Another well-known process, often referred to as a “raw starch hydrolysis” process (RSH process), includes grinding the starch-containing material and then simultaneously saccharifying and fermenting granular starch below the initial gelatinization temperature typically in the presence of an acid fungal alpha-amylase and a glucoamylase.

In a process for producing ethanol from corn, following SSF or the RSH process the ethanol is distilled from the whole mash after fermentation. The resulting ethanol-free slurry, usually referred to as whole stillage, is separated into solid and liquid fractions (i.e., wet cake and thin stillage containing about 35 and 7% solids, respectively). The thin stillage is often condensed by evaporation into a thick stillage or syrup and recombined with the wet cake and further dried into distillers' dried grains with solubles distillers' dried grain with solubles (DDGS) for use in animal feed.

In an embodiment of the present disclosure the xylanase may preferably be of microbial origin, such as of fungal origin (e.g., Aspergillus, Fusarium, Humicola, Meripilus, Trichoderma) or from a bacterium (e.g., Bacillus). In a preferred embodiment the xylanase is derived from a filamentous fungus, preferably derived from a strain of Aspergillus, such as Aspergillus aculeatus, or a strain of Humicola, preferably Humicola lanuginosa. Examples of xylanases useful in the methods of the present invention include, but are not limited to, Aspergillus aculeatus xylanase (GeneSeqP:AAR63790; WO 94/21785), Aspergillus fumigatus xylanases (WO 2006/078256), and Thielavia terrestris NRRL 8126 xylanases (WO 2009/079210). The xylanase may preferably be an endo-1,4-beta-xylanase, more preferably an endo-1,4-beta-xylanase of GH 10 or GH 1 1. Examples of commercial xylanases include SHEARZYME™, BIOFEED WHEAT™, HTec and HTec2 from Novozymes A/S, Denmark.

Examples of beta-xylosidases useful in the methods of the present invention include, but are not limited to, Trichoderma reesei beta-xylosidase (UniProtKB/TrEMBL accession number Q92458), Talaromyces emersonii (SwissProt accession number Q8X212), and Neurospora crassa (SwissProt accession number Q7SOW4).

Examples of suitable bacterial xylanases include xylanases derived from a strain of Bacillus, such as Bacillus subtilis, such as the one disclosed in U.S. Pat. No. 5,306,633 or others.

Contemplated commercially available xylanases include SHEARZYM E™, BIOFEED WHEAT™, (from Novozymes AJS), Econase CE™ (from AB Enzymes), Depol 676™ (from Biocatalysts Ltd.) and SPEZYME™ CP (from Genencor Int.

Xylanase may be added in an amount effective in the range from 0.16×10⁶-460×10⁶ Units per ton beer mash or fermentation medium.

Example of determination of Xylanase Activity (FXU):

The endoxylanase activity is determined by an assay, in which the xylanase sample is incubated with a remazol-xylan substrate (4-O-methyl-D-glucurono-D-xylan dyed with Remazol Brilliant Blue R, Fluka), pH 6.0. The incubation is performed at 50° C. for 30 min. The background of non-degraded dyed substrate is precipitated by ethanol. The remaining blue colour in the supernatant is determined spectrophotometrically at 585 nm and is proportional to the endoxylanase activity.

The endoxylanase activity of the sample is determined relatively to an enzyme standard.

The pectinase used in the methods according to the present disclosure may be any pectinase, in particular of microbial origin, in particular of bacterial origin, such as a pectinase derived from a species within the genera Bacillus, Clostridium, Pseudomonas, Xanthomonas and Erwinia, or of fungal origin, such as a pectinase derived from a species within the genera Trichoderma or Aspergillus, in particular from a strain within the species A. niger and A. aculeatus. Contemplated commercially available pectinases include Pectinex Ultra-SPL™ (from Novozymes), Pectinex Ultra Color (from Novozymes), Rohapect Classic (from AB Enzymes), Rohapect 10L (from AB Enzymes). Pectinase may be added in an amount effective in the range from 1.4×10⁹-23500×10⁹ Units per ton beer mash or fermentation medium.

Example of determination of Pectintranseliminase Unit (PECTU):

The method is based on the enzyme's degradation of a pectin solution by a transeliminase reaction, the double bonds formed result in an increase in the absorption at 238 nm which is followed by a spectrophotometer.

### Reaction Conditions

Temperature: 30° C.±0.5° C.
pH: 3.50±0.02
Substrate: 0.24% Pectin (Ohipektin, Brown Ribbon Pure, Art. no. 1.1B00.A. Lot no. 0304)
Enzyme concentration: 1.9-2.3 PECTU/mL
Reaction time: 6 minutes
Measuring time: 5 minutes
Wavelength: 238 nm

The activity is determined relative to a PECTU standard. The result is given in the same units as for the standard, which is designated; PECTU—Pectintranseliminase Unit.

The term “alpha-amylase” means an alpha-1,4-glucan-4-glucanohydrolase (E.C. 3.2.1.1) that catalyzes the hydrolysis of starch and other linear and branched 1,4-glucosidic oligo- and polysaccharides.

In an embodiment, the xylanase may be added in an amount of 1-30, e.g., 5-30 7-25, 10-20, 10-17, or 12-15 micrograms/g dry solids.

In an embodiment, the pectinase may be added in an amount of 0.01-1.0, e.g., 0.015-0.08, 0.015-0.06, 0.015-0.04, or 0.02-0.03 FXU/g dry solids.

The saccharification and fermentation steps may be carried out either sequentially or simultaneously. The xylanase and the pectinase may be added during saccharification and/or after fermentation when the process is carried out as a sequential saccharification and fermentation process and before or during fermentation when steps (b) and (c) are carried out simultaneously (SSF process).

As mentioned above, the fermenting organism is preferably yeast, e.g., a strain of Saccharomyces cerevisiae or Saccharomyces diastaticus. In an avantegeous embodiment a yeast strain of Saccharomyces diastaticus is used (SIHA Amyloferm®, E. Begerow GmbH&Co, Langenlonsheim, Germany) since their exo-amylase activity can split liquid starch and also dextrin, maltose and melibiose.

In the liquefaction step the gelatinized starch (downstream mash) is broken down (hydrolyzed) into maltodextrins (dextrins). To achieve starch hydrolysis a suitable enzyme, preferably an alpha-amylase, is added. Liquefaction may be carried out as a three-step hot slurry process. The slurry is heated to between 60-95° C., preferably 80-85° C., and an alpha-amylase may be added to initiate liquefaction (thinning). Then the slurry may be jet-cooked at a temperature between 95-140° C., preferably 105-125° C., for about 1-15 minutes, preferably for about 3-10 minutes, especially around about 5 minutes. The slurry is cooled to 60-95° C. and more alpha-amylase may be added to complete the hydrolysis (secondary liquefaction). The liquefaction process is usually carried out at a pH of 4.0 to 6.5, in particular at a pH of 4.5 to 6.

The saccharification step and the fermentation step may be performed as separate process steps or as a simultaneous saccharification and fermentation (SSF) step. The saccharification is carried out in the presence of a saccharifying enzyme, e. g. a glucoamylase, a beta-amylase or maltogenic amylase. Optionally a phytase and/or a protease is added.

Saccharification may be carried out using conditions well known in the art with a saccharifying enzyme, e.g., beta-amylase, glucoamylase or maltogenic amylase, and optionally a debranching enzyme, such as an isoamylase or a pullulanase. For instance, a full saccharification process may last up to from about 24 to about 72 hours, however, it is common to do a pre-saccharification for typically 40-90 minutes at a temperature between 30-65° C., typically about 60° C., followed by complete saccharification during fermentation in a simultaneous saccharification and fermentation process (SSF process). Saccharification is typically carried out at a temperature from 20-75° C., preferably from 40-70° C., typically around 60° C., and at a pH between 4 and 5, normally at about pH 4.5.

The most widely used process to produce a fermentation product, especially ethanol, is the simultaneous saccharification and fermentation (SSF) process, in which there is no holding stage for the saccharification, meaning that a fermenting organism, such as a yeast, and enzyme(s), including the hemicellulase(s) and/or specific endoglucanase(s), may be added together. SSF is typically carried out at a temperature from 25° C. to 40° C., such as from 28° C. to 35° C., from 30° C. to 34° C., preferably around about 32° C. In an embodiment, fermentation is ongoing for 6 to 120 hours, in particular 24 to 96 hours.

During and/or after the fermentation, the fermented mash is subjected to an enzyme composition according to the present disclosure. In an embodiment, the enzyme composition comprises a xylanase and a pectinase.

In a particular embodiment, the process of the present disclosure further comprises, prior to liquefying the starch-containing material the steps of:
reducing the particle size of the starch-containing material, preferably by milling; and
forming a slurry comprising the starch-containing material and water.

The aqueous slurry may contain from 10-55 w/w % dry solids (DS), preferably 25-45 w/w % dry solids (DS), more preferably 30-40 w/w % dry solids (DS) of the starch-containing material. The slurry is heated to above the gelatinization temperature and an alpha-amylase, preferably a bacterial and/or acid fungal alpha-amylase, may be added to initiate liquefaction (thinning). The slurry may be jet-cooked to further gelatinize the slurry before being subjected to an alpha-amylase in step (a).

In a preferred embodiment, the starch containing material is milled cereals, preferably barley or corn, and the methods comprise a step of milling the cereals before step (a). In other words, the disclosure also encompasses methods, wherein the starch containing material is obtainable by a process comprising milling of cereals, preferably dry milling, e. g. by hammer or roller mils. Grinding is also understood as milling, as is any process suitable for opening the individual grains and exposing the endosperm for further processing. Two processes of milling are normally used in alcohol production: wet and dry milling. The term “dry milling” denotes milling of the whole grain. In dry milling the whole kernel is milled and used in the remaining part of the process Mash formation. The mash may be provided by forming a slurry comprising the milled starch containing material and brewing water. The brewing water may be heated to a suitable temperature prior to being combined with the milled starch containing material in order to achieve a mash temperature of 45 to 70° C., preferably of 53 to 66° C., more preferably of 55 to 60° C. The mash is typically limited in a tank known as the slurry tank.

Subsequent to fermentation the fermentation product may be separated from the fermentation medium. The slurry may be distilled to extract the desired fermentation product or the desired fermentation product from the fermentation medium by micro or membrane filtration techniques. Alternatively the fermentation product may be recovered by stripping. Methods for recovering fermentation products are well known in the art. Typically, the fermentation product, e.g., ethanol, with a purity of up to, e.g., about 96 vol. % ethanol is obtained.

Following the completion of the fermentation process, the material remaining is considered the whole stillage. As used herein, the term “whole stillage” includes the material that remains at the end of the fermentation process both before and after recovery of the fermentation product, e.g., ethanol. The fermentation product can optionally be recovered by any method known in the art. In one embodiment, the whole stillage is separated or partitioned into a solid and liquid phase by one or more methods for separating the thin stillage from the wet cake. Such methods include, for example, centrifugation and decanting. The fermentation product can be optionally recovered before or after the whole stillage is separated into a solid and liquid phase.

Thus, in one embodiment, the methods of the disclosure further comprise distillation to obtain the fermentation product, e.g., ethanol. The fermentation and the distillation may be carried out simultaneously and/or separately/sequentially; optionally followed by one or more process steps for further refinement of the fermentation product.

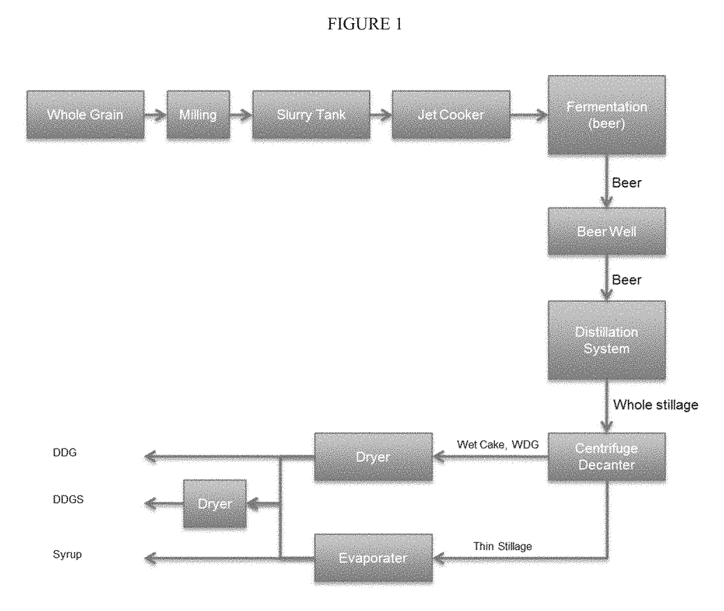

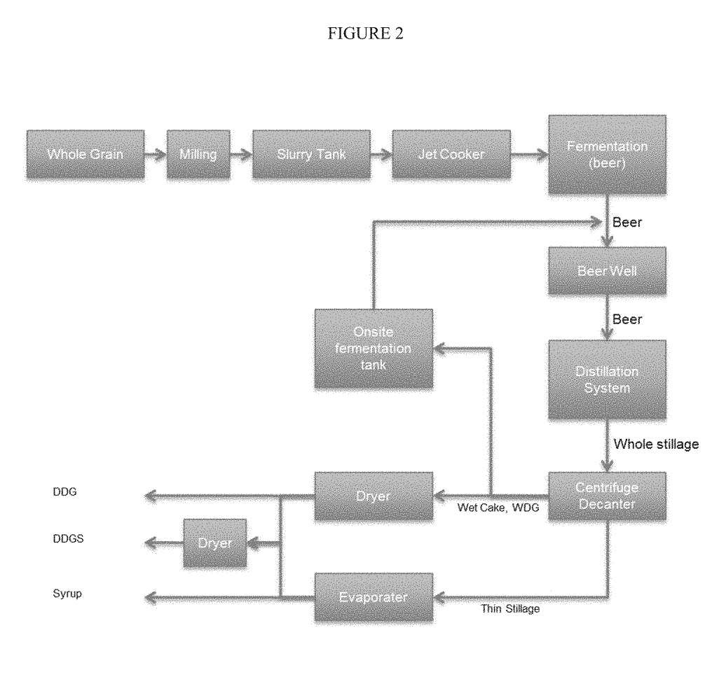

In an embodiment, the aqueous by-product (whole stillage) from the distillation process is separated into two fractions, e.g., by centrifugation: wet grain (solid phase), and thin stillage (supernatant). In another embodiment, the methods of the disclosure further comprise separation of time whole stillage produced by distillation into wet grain and thin stillage; and recycling thin stillage to the starch containing material prior to liquefaction. In one embodiment, the thin stillage is recycled to the milled whole grain slurry. The wet grain fraction may be dried, typically in a drum dryer. The dried product is referred to as distillers dried grains, and can be used as mentioned above as high quality animal feed. The thin stillage fraction may be evaporated providing two fractions (see FIG. 1 and FIG. 2), (i) a condensate fraction of 4-6% DS (mainly of starch, proteins, oil and cell wall components), and (ii) a syrup fraction, mainly consisting of limit dextrins and non-fermentable sugars, which may be introduced into a dryer together with the wet grains (from the whole stillage separation step) to provide a product referred to as distillers dried grain with solubles, which also can be used as animal feed. Thin stillage is the term used for the supernatant of the centrifugation of the whole stillage. Typically, the thin stillage contains 4-6% DS (mainly starch and proteins) and has a temperature of about 60-90° C. In another embodiment, the thin stillage is not recycled, but the condensate stream of evaporated thin stillage is recycled to the slurry containing the milled whole grain to be jet cooked.

Further details on how to carry out liquefaction, saccharification, fermentation, distillation, and recovering of ethanol are well known to the skilled person.

Methods for dewatering stillage and for extracting oil from a fermentation product are known in the art. These methods include decanting or otherwise separating the whole stillage into wet cake and thin stillage. See, e.g., U.S. Pat. Nos. 6,433,146, 7,601,858, and 7,608,729, and U.S. Application Publication No. 2010/0058649. Furthermore, the thin stillage can be evaporated or condensed into syrup or thick stillage from which the oil can be extracted utilizing centrifugation, filtering, heat, high temperature, increased pressure, or a combination of the same. Another way to extract oil is to lower the pH of the thin stillage or syrup. The use of surfactants to break emulsions also enhances oil extraction. Presses can also be used for dewatering. In one embodiment of the disclosure, the presence of pectinase and xylanase in the fermented mash after the fermentation increases the amount of oil in the thin stillage and further the syrup or thick stillage.

The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation as mentioned above. Ethanol with a purity of up to about 96 vol. % can be obtained, which can be used as, for example, fuel ethanol, drinking ethanol, i.e., potable neutral spirits, or industrial ethanol.

In accordance with the purposes of the present invention as described herein, in one aspect of the present disclosure a method is provided for improving the nutritional quality of a by-product or residue of a fermentative production process, comprising inoculating the by-product or residue with at least one filamentous fungus, fermenting the by-product or residue and separating at least one enzyme from the fermented by-product or residue; and providing the enzyme to fermented mash (beer mash) of an fermentative production process, preferably an ethanol production process. The filamentous fungus may be selected from the group consisting of Rhizopus, Aspergillus, Trichoderma, and any combination thereof. The by-product or residue is preferably a fibrous by-product and may be selected from the group consisting of spent brewer's grains, dried distiller's grains, dried distiller's solubles, distiller's dried grains with solubles and WDG, and mixtures thereof.

Some embodiments of the present disclosure pertain to methods for the manufacturing of an enzymes composition used for treating fermented mash of in an fermentative production process to improve the nutritional quality of a by-product or residue and/or the process ability of the production process, comprising:
a) inoculating the by -product or residue with at least one filamentous fungus;
b) fermenting the by-product or residue; and
c) separating at least one enzyme from the fermented by-product or residue, wherein
   the filamentous fungus may be selected from the group consisting of Rhizopus, Aspergillus, Trichoderma, and any combination thereof.

In some embodiments, the by-product or residue can be a fibrous by-product selected from the group consisting of spent brewer's grains, dried distiller's grains, dried distiller's soluble, distiller's dried grains with soluble, wet grains, and mixtures thereof.

The inventions described and claimed herein are not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present invention is further described by the following examples which should not be construed as limiting the scope of the invention.

### EXAMPLES

### a) Increased Oil Production

The oil content of DDGS is sometimes higher than desired and methods of recovering more oil as a separate by-product for use in biodiesel production or other bio renewable products are sought. Much of the work in oil recovery from fermentation processes has focused on improving the extractability of the oil from. the whole stillage. As mentioned in the specification, a better de-oiling leads to a better oil separation within the ethanol process. The corn oil production is a high valuable byproduct for the food and feed industry also for the Biodiesel production.

For that example the de oiling capability of the whole stillage during the centrifugation was tested and determined.

Four different setups were tested.

In the first fermentor (Fermentor #1) the fermentation cultivation was not treated with enzymes (0 g/t xylanase; 0 g/t pectinase), in the second, third and forth fermentor (Fermentor #2 to #4) the fermentation cultivation was treated with the enzyme composition comprising a xylanase and a pectinase from the beginning of the fermentation in different concentrations (from 25 g/t to 200 g/t xylanase and from 25 g/t to 200 g/t pectinase).

The trials were performed in 2 L fermentation scale. After the distillation the fermenter samples with the whole stillage were taken and the whole stillage was centrifuged to obtain thin stillage and the wet cake. Oil extraction was carried out by hexane and the mass of the extracted oil was related to the volume of thin stillage.

In one embodiment, the process of the production of ethanol from corn was performed as follows:

### A) Process For Producing Fermentation Products

### a) Reducing the Particle Size of the Starch-Containing Material by Milling

corn (Company Pannonia, Hungary) was milled to <2 mm particle size (coffee mill, company Brunn)

### b) Forming a Slurry Comprising the Starch-Containing Material and Water

1.5 kg milled corn was added to 4.96 L ml warm tap water (water hardness 3.57 mmol/L) at 35° C. to obtain a 25% solid solution with a final volume of 6 L in a Biostat C fermentor (company Sartorius) leading to a pH of about 5.6.

### c) Liquefying of the Starch-Containing Material

temperature was increased to 90° C.
1 ml α-amylase “α-amylase VF-Kartoffel” (Schliessmann, Nr. 5049) was diluted in 10 ml tap water and then the diluted amylase was added to the slurry
temperature was increased to 90° C.
the fermentor was incubated for 90 min at 90° C. and 450 rpm
the slurry was cooled to 30° C., pH was adjust to ˜4 with 30% H₂SO₄

### d) Saccharifying of the Liquefied Material Obtained

1.5 ml glucoamylase “Amylase GA 500” (Schliessmann, Nr. 5042) was diluted in 10 ml sterile tap water and then the diluted glucoamylase was added to the slurry, which is the saccharified liquefied material.

### e) Fermentation

1.8 g (NH4)2SO4 (i.e. 300 ppm ammonium sulphate) was added to the 6 L saccharified liquefied material.
the saccharified liquefied material containing 300 ppm ammonium sulphate in the Biostat C fermentor was stirred with 800 rpm for 5 min to distribute everything evenly.
the saccharified liquefied material containing 300 ppm ammonium sulphate (i.e. mash) was distributed in 1500 g single portions into four 2 L Biostat B fermentors (company Sartorius) containing a horseshoe mixer.
Enzyme stock preparation: 2.5 g of the pectinase (Pec3) with 90349 U/mL and 2.5 g of the xylanase (Xy116) with 10027 U/mL were added into a 50 mL graduated cylinder and filled to 50 mL with tap water.

The enzyme stock was transferred into a 50 mL tube and then stored at 4° C. until use within one hour.
The following volumes of the enzyme stock preparation were added to the 2 L Biostat B fermentor containing 1500 g of the saccharified liquefied material containing 300 ppm ammonium sulphate.

Fermentor #1: 0 mL of the enzyme stock preparation leading to 0 g/t of pectinase and 0 g/t of xylanase

Fermentor #2: 0.75 mL of the enzyme preparation leading to 25 g/t of pectinase and 25 g/t of xylanase

Fermentor #3: 2.25 mL of the enzyme preparation leading to 75 g/t of pectinase and 75 g/t of xylanase

Fermentor #4: 6.00 mL of the enzyme preparation leading to 200 g/t of pectinase and 200 g/t of xylanase
Yeast propagation: 300 ml autoclaved YNB (yeast nitrogen base) medium plus glucose with 10 g/L glucose medium resulting in pH 5.7 in a 1 L cultivation flasks, which had been inoculated with 2 ml yeast (Ethanol RED, company Fermentis) from a −80° C. cryo stock containing 20% glycerol, were incubated for 23 hours (30° C., 150 rpm) leading to the yeast culture.
Each of the four fermentors (Fermentor #1 to #4) was inoculated by 60 mL of the yeast culture.
Cultivations of the fermentors were carried out at 30° C. at 150 rpm, without pH control for 92.5 hours.
7-ml samples were taken twice the day by cut off pipette to monitor fermentation progress (ethanol concentration). The samples were transferred in 15 mL tubes and centrifuged at 4470 g for 10 minutes at 4° C. and stored until further analysis at −20° C.

### f) Ending the Fermentation Process and Distillation of Ethanol

To end the fermentation process and to distillate ethanol the four fermentors were incubated at 80° C. for 20 min and stirred at 150 rpm.

### g) Taking Samples For Analysis of Oil Release

After the 20 min at 80° C. and stirring at 150 rpm 3×40 to 45 ml samples were taken from each fermentor by a 50 mL pipette and filled into 50 mL tubes (samples of whole stillage).

### B) Enzyme Product Activity Determination:

DNSA solution: For the DNSA solution the following compounds were used:
5.00 g 3.5-Dinitrosalicylic acid (DNSA) was dissolved in 300 ml distilled H₂O.
add 50 ml NaOH/KOH-solution (4M KOH+4 M NaOH) drop per drop
add 150 g K—Na-tartrate tetrahydrate
cool solution to room temperature
ad with destined H₂O to 500 ml final volume
store in the darkness

### a) Pectinase

Substrates: Polygalacturonic acid (Sigma 81325)

Substrates were dissolved in buffer to a concentration of 0.8% (w/v)

Buffer: 50 mM sodium acetate, pH 4.5

For the assay 96 well PCR microtiter plates (company Greiner) were used. The enzymes were diluted in buffer. 90 μl substrate and 10 μl enzyme solution were mixed. A blank was measured replacing enzyme solution with water. Incubation was carried out for 30 min at 37° C., followed by a 5 minute enzyme inactivation step at 99° C. and followed by cooling for 10 min at 4° C. In a second 96 well PCR microtiter plates (company Greiner) 50 μl of the incubated substrate—enzyme mix was incubated with 50 μl of the DNSA solution at 98° C. for 10 minutes and then cooled to 4° C. and incubated for 5 minutes at 4° C.

100 μl of the reaction was transferred into a well of 96 well transparent, flat bottom micro titer plate and the adsorption was measured at 540 nm by a micro titer plate reader (Tecan M1000).

### b) Xylanase

Substrates: Xylan from Birchwood (Sigma X0502)

Substrate was dissolved in buffer to a concentration of 1.5% (w/v)

Buffer: 100 mM sodium acetate, pH 5.0 containing 20 mM CaCl₂ and 0.4 g/L Tween20

For the assay 96 well PCR microtiter plate (company Greiner) were used. The enzymes were diluted in buffer. 90 μl substrate and 10 μl enzyme solution were mixed. A blank was measured replacing enzyme solution with water. Incubation was carried out for 20 min at 40° ° C. followed by a 5 minute enzyme inactivation step at 99° C. and followed by cooling for 5 min at 4° C. 45.5 μL of the DNSA solution was added to the 96 well PCR microtiter plate by a multidrop (company Fisher-Scientific) and then the plate was incubated for 98° C. for 10 minutes and cooled to 4° C. and incubated for 5 minutes at 4° C.

100 μl of the reaction was transferred into well of a new 96 well transparent, flat bottom micro titer plate and then the adsorption was measured at 540 nm by a micro titer plate reader (Tecan M1000).

The activity is calculated as Units per μl or mg of enzyme product. 1 unit is defined as the amount of formed reducing ends in μmol per minute. The enzyme activities are shown in Table 1.

TABLE 1

| Type | Activity | Shortname |
|---|---|---|
| pectinase | 90349 U/mL | Pec3 |
| xylanase | 10027 U/mL | Xyl16 |

### Example 1

### Analysis of De-Oiling

After the fermentation process and distillation of ethanol the following steps were made to analyze de-oiling:
1. About 40 ml of the samples of whole stillage were transferred into a 50 mL Falcon tube (FT1) and the mass was determined (mass FT1sample). The mass of the empty FT1 was previously determined (mass FT1empty). FT1 with the sample (FT1sample) was centrifuged at 3000 g for 30 minutes.
2. The supernatant was decanted into a new 50 mL Falcon tube (FT2) leading to the thin stillage and the mass was determined (mass FT2sample). The mass of FT2 was previously determined (mass FT2empty).
3. Falcon tubes FT1 and FT2 were stored at 4° C., until further analysis
4. Falcon tubes FT1 and FT2 were warmed to 20° C. in a water bath
5. To the sediment in FT1 5 mL hexane was added, FT1 was washed with it and the liquid was poured into a round bottom flask (RBF1), which mass was previously determined (mass RBF1empty)
6. 10 mL of hexane was added to the supernatant in FT2 and shaken vigorously for two minutes.
7. FT2 was centrifuged at 3000 g for 10 minutes and the upper phase was added to RBF1
8. 2 mL of hexane was rinsed into FT2 and then added to RBF1
9. Steps 6 to 7 were repeated, but liquid was added into a new round bottom flask (RBF2) (mass RBF2empty).
10. Hexane in RBF1 and RBF2 was evaporated in a rotary evaporator at 250 mbar at 90° C.
11. RBF1 and RBF2 were dried at 90° C. for 12 hours.
12. RBF1 and RBF2 were cooled to room temperature and then the mass of RBF1 (mass RBF1oil) and RBF2 (mass RBF2oil) containing corn oil was determined
   Calculation of extracted oil:

Mass of extracted oil [mg]=(mass RBF1oil−mass RBF1empty)+(mass RBF2oil−mass RBF2empty)

Calculation of sample mass:

Mass of thin stillage [mg]=(mass FT2sample−mass FT2empty)

Calculation of extracted oil per mass thin stillage

Extracted oil mass per mass of thin stillage [mg oil/mg thin stillage]=mass of extracted oil [mg]/mass of thin stillage [mg]

Calculation of extracted oil per volume thin stillage

Extracted oil mass per mass of thin stillage [mg oil/mg thin stillage]=was calculated to extracted oil mass per mass of thin stillage [mg oil/kg thin stillage].

With the assumption that 1 kg thin stillage has 1 L volume:

Extracted oil mass per mass of thin stillage [mg oil/kg thin stillage] was converted to extracted oil per volume of thin stillage [mg oil/L thin stillage]

After the reaction of the enzyme composition comprising the main activities xylanase and pectinase with the concentrations 25 g/t, 75 g/t and 200 g/t of each compound followed by the centrifugation (conditions: 3000 rpm for 30 min.) of the whole stillage an improved oil separation in the thin stillage was observed.

Table 2 is showing the extracted oil after centrifugation.

TABLE 2

| | | Extracted oil per | |
|---|---|---|---|
| | | volume of thin | |
| | | stillage | |
| added | added | [mg oil/L thin | Related to control |
| xylanase [g/t] | pectinase [g/t] | stillage] | [0 g/t = 100%] in [%] |
| 0 | 0 | 993 | 100 |
| 25 | 25 | 1116 | 112 |
| 75 | 75 | 1804 | 182 |
| 200 | 200 | 2272 | 228 |

The whole stillage, in which the inoculated mash was treated from the beginning of the fermentation with the enzyme composition comprising (Table 1) the main activities xylanase and pectinase activity, showed a higher extraction of oil per volume of thin stillage compared to the inoculated mash, which was not treated with enzymes.

### Further Examples

### A) Processes For Producing Fermentation Products

In one embodiment, the process of the production of ethanol form corn was performed as follows:

### a) Reducing the Particle Size of the Starch-Containing Material by Milling:

corn was milled to <2 mm particle size

### b) Forming a Slurry Comprising the Starch-Containing Material and Water

10 kg corn were tap water at 35° C. to obtain a ˜31.25% solid solution, total volume 30 liter
pH range was 5.6-6.0

### c) Liquefying of the Starch-Containing Material

temperature was increased to 90° C.
7 ml alpha-amylase (Novozymes Liquozyme SC) were added
1% antifoam was added (30 ml)
incubation for 90 min at 90° C. and 150 rpm
slurry was cooled to 30° C., pH adjusted to ˜4 with 1 NH₂SO₄

### d) Saccharifying of the Liquefied Material Obtained

12 ml Glucoamylase (Novozymes Spirizyme Ultra) was added

### e) Fermentation

Yeast propagation: 200 ml YNB-starch medium was incubated overnight (30° C., 150 rpm) and inoculated with 2 g yeast (SIHA Amyloferm), the complete pre-culture was added to the fermentation
addition of 300 ppm ((NH4)2SO4) (10 g) as nitrogen source
yeast addition
pH was titrated to 4.0 with ammoniac solution (25%), supplies further nitrogen
incubation for max. 48 h at 30° C. and 100 rpm
2-ml samples were taken every 12 hrs to monitor fermentation progress (sugar-, ethanol concentration)

### B) Enzymes:

The following enzymes listed in Table 3 were used alone or in different combinations.

TABLE 3

| Typ | Activity | Shortname |
|---|---|---|
| Cellulase | 565.84 U/g | Cel2 |
| endo-1,4 beta | 6538.01 U/g | Man3 |
| Mannanase | | |
| Pectinase | 90349.55 U/ml | Pec3 |
| β 1-3 Glucanase | 10016.97 U/g | Glu1 |
| Xylanase | 34094.02 U/g | Xyl4 |
| Xylanase | 35094.00 U/g | Xyl7 |

### C) Enzyme Product Activity Determination:

Substrates: Glucanase: β-Glucan from barley, low viscosity (Megazyme)
Xylanase: Xylan from Birchwood (Sigma)
Mannanase: Galactomannan, carob
Pectinase: Polygalacturonic acid (Sigma)

Substrates were dissolved in buffer to a concentration of 0.8% (w/v)

### Buffer: 50 mM NaAcetat, pH 4.5

The Enzymes were diluted in buffer, the right enzyme concentration must be determined for each enzyme. A 90 μl substrate and 10 μl enzyme solution were mixed. A blank was measured replacing enzyme solution with water. Incubation for 30 min at 37° C. (followed from 10 min at 4° C.). Reducing sugars were measured after mixing of 50 μl of the incubated substrate—enzyme mix with 50 μl DNSA-Reagent.

The activity is calculated as Units per μl or mg of enzyme product. 1 unit is defined as the amount of formed reducing ends in μmol per minute. One protease unit is defined as the formation of glycin equivalents per minute. The enzyme activities are shown in Table 4.

TABLE 4

| | Main | |
|---|---|---|
| Shortname | Activity | Unit |
| Cel1 | 79.64 | U/ml |
| Cel2 | 565.84 | U/g |
| Cel3 | 1061.00 | U/ml |
| Man1 | 6205.96 | U/g |
| Man2 | 7988.38 | U/g |
| Man3 | 6538.01 | U/g |
| Pec1 | 28242.70 | U/ml |
| Pec2 | 37473.18 | U/ml |
| Pec3 | 90349.55 | U/ml |
| Pec4 | 117283.35 | U/ml |
| Pec5 | 44082.56 | U/ml |
| Glu1 | 10016.97 | U/g |
| Glu2 | 10594.57 | U/g |
| Glu3 | 3583.18 | U/g |
| Glu5 | 763.00 | U/ml |
| Xyl1 | 2264.56 | U/ml |
| Xyl2 | 4434.68 | U/g |
| Xyl3 | 314.83 | U/g |
| Xyl4 | 34094.02 | U/g |
| Xyl5 | 4.28 | U/g |
| Xyl7 | 35094.00 | U/g |
| Xyl8 | 14108.00 | U/g |
| Xyl9 | 75000.00 | U/g |
| Xyl10 | 790.00 | U/g |
| Xyl11 | 1178.00 | U/g |
| Xyl12 | 16603.50 | U/g |

### Example 1

### Dewatering of Whole Stillage

Beer (8.7 wt-% dry solids, pH=4.0 from conventional, dry-milled ethanol fermentation was used as substrate.

An aliquot (50 mL) of whole stillage was placed into a centrifuge tube and warmed to 37<0>C. Total enzyme concentration added was 200 ppm, the mixture was gently agitated overnight on a rotary shaker.

The tube was centrifuged for 5 minutes at 2000 rpm. The supernatant was decanted and the resulting wet cake was weighed and compared to the control without addition of any enzyme. The results, along with several enzymes and enzyme combinations tested, are shown in Table 5 and Table 6.

TABLE 5

| Enzyme | wet cake | StdDev |
|---|---|---|
| Cel1 | 91.9% | 1.15 |
| Cel2 | 90.8% | 1.06 |
| Man1 | 87.8% | 4.01 |
| Man2 | 86.7% | 2.53 |
| Man3 | | |
| Men1 | 99.6% | 0.4 |
| Pec1 | 97.8% | 2.48 |
| Pec2 | 87.1% | 8.16 |
| Pec3 | 80.8% | 0.71 |
| Pec4 | 82.0% | 0.57 |
| Pec5 | 94.6% | 3.65 |
| Pro1 | 87.8% | 1.12 |
| Pro2 | 96.2% | 0.89 |
| Pro3 | 93.7% | 1.34 |
| Pro4 | 91.5% | 1.97 |
| Pro5 | 93.3% | 4.77 |
| Glu1 | 85.8% | 1.33 |
| Glu2 | 79.6% | 0.81 |
| Glu3 | 90.3% | 0.65 |
| Xyl1 | 79.4% | 1.29 |
| Xyl2 | 91.3% | 1.17 |
| Xyl3 | 86.1% | 1.16 |
| Xyl4 | 77.0% | 0.92 |
| Xyl5 | 90.1% | 0.41 |
| Xyl6 | 89.6% | 2.05 |

TABLE 6

| Enzyme combinations | Wet cake | StdDev |
|---|---|---|
| Xyl4 | 87.4% | 0.23 |
| Xyl4 + Glu1 | 83.1% | 0.26 |
| Xyl4 + Pec3 | 86.7% | 0.63 |
| Xyl4 + Man3 | 82.7% | 0.30 |
| Xyl4 + Cel2 | 88.3% | 0.05 |
| Xyl4 + Pro1 | 87.4% | 0.58 |
| Xyl4 + Glu1 + Prot1 | 78.5% | 0.07 |
| Xyl4 + Pec3 + Prot1 | 82.6% | 0.20 |
| Xyl4 + Man3 + Prot1 | 82.5% | 0.13 |
| Xyl4 + Cel2 + Prot1 | 82.7% | 0.19 |
| Xyl4 + Pec3 + Man3 | 82.7% | 0.19 |
| Xyl4 + Cel2 + Man3 | 83.2% | 0.11 |
| Xyl4 + Cel2 + Pec3 | 82.6% | 0.13 |
| Xyl4 + Pec3 + Glu1 | 81.4% | 0.04 |

### Example 2

### Fiber Reduction in DDGS After Enzymatic Treatment Measured by Determination of ADF and NDF

Acid Detergent Fibers (ADF) and Neutral Detergent Fibers (NDF) are dietary fibers and can be soluble and insoluble. ADF containing lignin and cellulose. NDF is containing ADF and hemicelluloses. Soluble fibers are responsible for viscosity effects within the digestion process and responsible for reduce energy and protein uptake (cage effect). Reduced ADF/NDF values leads to better digestion within the intestinal due to the reduced viscosity in coincidence with better protein and energy release.

For the determination of Acid Detergent Fibers (ADF) and Neutral Detergent Fibers (NDF) feed industry wide analytical methods are used. (VDLUFA Bd. III, 6.5.2)

Beer (10 wt-% dry solids, pH=4.0) from conventional, dry-milled ethanol fermentation was used as substrate.

An aliquot (50 mL) of beer was placed into a centrifuge tube and heated to 37° C. In the added enzyme composition, the total enzyme amount was in the range of 100 ppm to 400 ppm, the mixture was gently agitated for 6 hours on a rotary shaker. After thermal enzyme inactivation (1 hour at 80° C.) the whole stillage was dried and milled. The dried stillage was analyzed for ADF/NDF according the protocols of VDLUFA Bd. III, 6.5.2. (Method book III “The chemical analysis of feedstuff” of VDLUFA 1st-7th supplement delivery)

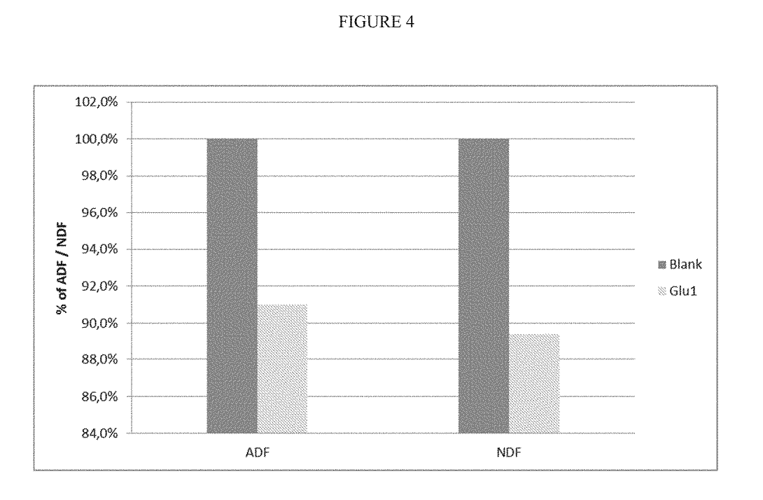

The results are shown FIGS. 4 to 7. FIG. 4 shows the result of adding an enzyme composition comprising beta-1,3-glucanase as the main activity (Rohalase BX, AB Enzymes). The measured parameters are Neutral Detergent Fibers (NDF) and Acid Detergent Fibers (ADF). The enzyme dosage was 200 g/t of beer. These results shows clearly that the β-1,3-glucanase enzyme reduces the values of ADF and NDF by 9% and 10.6%.

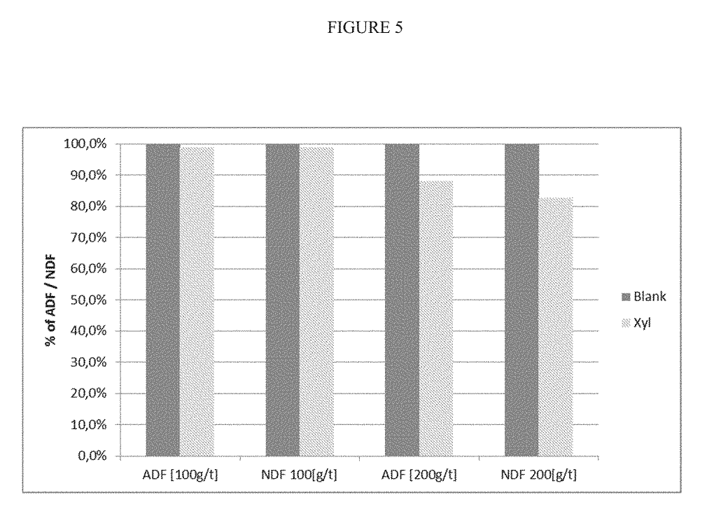

FIG. 5 shows the effect of an enzyme composition comprising a xylanase as the main activity (Xylanase 2 XP Conc, Dyadic) applied in two concentrations of 100 and 200 g/t of beer. The xylanase reduces the fiber content of DDGS which can be seen in the clear reduction of ADF and NDF. With an enzymes dosage of 100 g/t the NDF concentration is only reduced by 1.2% whereas the reduction was 17.3% for a dosage of 200 g/t.

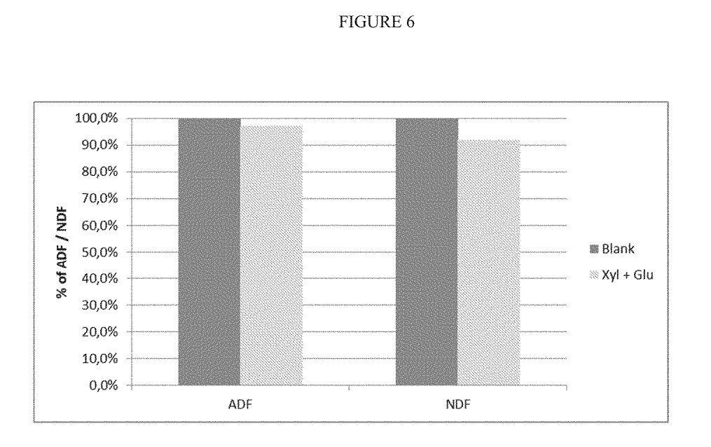

FIG. 6 shows the effect of an enzyme composition comprising as main activities xylanase and 1,3-glucanase activity (BLUZY-D, DIREVO Industrial Biotechnology GmbH), added in concentrations of 200 g/t beer.

Compared to FIG. 5 where xylanase used in a concentration of 100 g/t alone showed only minimal effect on fiber reduction, the combination of xylanase and 1,3-glucanase has a higher effect. The ADF value is reduced by 2.8% and the NDF value by 8.2%.

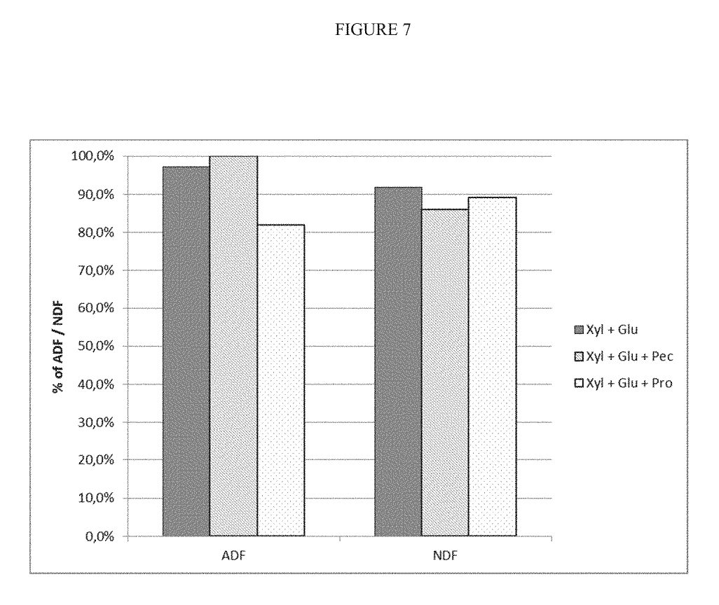

FIG. 7 shows the ADF/NDF reduction of the combination of xylanase and 1,3-glucanase in two combinations each with additional enzymes. An enzyme composition comprising in addition pectinase activity (Rohapect Classic, AB Enzymes) is showing an additional reduction of NDF (5.8%) but the enzyme is overriding the effect for ADF. The additional application of proteases is affecting the ADF and NDF reduction positive. The NDF value is reduced again by 3.2%.

### Example 3

### De-Oiling Improvement

The oil content of DDGS is sometimes higher than desired and methods of recovering more oil as a separate by-product for use in biodiesel production or other biorenewable products are sought. Much of the work in oil recovery from fermentation processes has focused on improving the extractability of the oil from the whole stillage. As mentioned in the specification, a better de-oiling leads to a better oil separation within the ETOH process. The corn oil production is a high valuable byproduct for the food and feed industry also for the Biodiesel production.

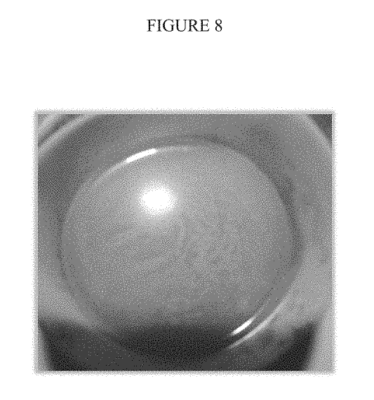

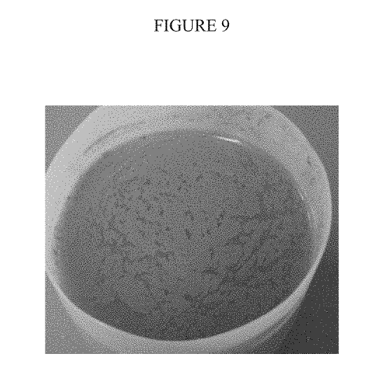

After the reaction of an enzyme composition comprising as main activities xylanase and 1,3-glucanase activity (BLUZY-D, DIREVO Industrial Biotechnology GmbH) with a concentration of 400 g/t beer, followed by the centrifugation (conditions: 3000 rpm for 10 min.) of the whole stillage an improved oil separation in the thin stillage was observed. FIGS. 8 and 9 are showing results of thin stillages after centrifugation. The thin stillage where the beer was treated with the enzyme composition comprising as main activities xylanase and 1,3-glucanase activity (FIG. 9) shows a much bigger oil layer on the supernatant compared to the thin stillage where the beer was not treated with enzymes (FIG. 8). FIG. 8 shows that no oil separation has been adjusted. FIG. 9 shows that a clear separation of oil, forming a thick oil layer can be observed.

### Example 4

### Feeding Tests

With the following animal trial it was shown that the exchange of expensive feed ingredients, like soybean meal, corn or wheat, with DDGS, especially DDGS that is modified during the production processes and methods according to the present disclosure, is possible and do not results in lower weight gains. To show the same performance regards weight gain between general feed stuff and feed stuff where parts are exchanged by DDGS is the goal of this test.
Usage of quails as tests animals
Feeding 10 cages with 2 animals for each treatment
DDGS inclusion in feed 20%
positive control was standard feed (wheat and soybean meal diet) without DDGS
All feed ratios were balanced to be isonitrogen (every feed contains the same amount of CP) and isocaloric (each feed contains the same amount of metabolized energy)
3 animal groups with normal feed, untreated DDGS and enzymatically optimized DDGS
Performance parameters: weight gain, Feed conversion ration

For the tests, two Different DDGS types were produced:
a) DDGS resulting from a treatment of the fermented mash in the ethanol production from A) of the present disclosure with an enzyme composition comprising a xylanase and a 1,3-glucanase (DDGS-treated)
b) DDGS produced without an enzyme treatment of the fermented mash (DDGS-blank).

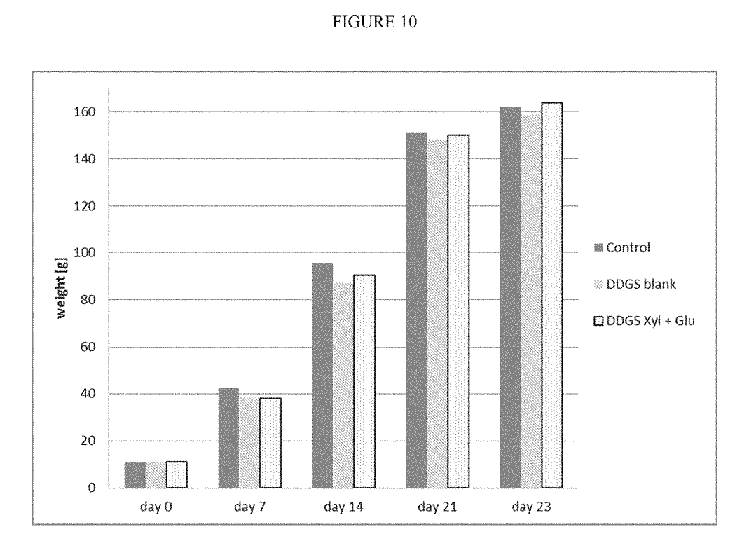

As a positive control a standard quail feed was used. FIG. 10 shows the results and the weight gain of quails fed with different feed stuff. In the starter phase (day 0-14) the normal feed leads to highest weight gain. In the grower phase, feed with 20% of the DDGS-treated, lead to the highest weight gain and outperformed the other two feeds. Based on the animal trial data, the results between the DDGS-blank and DDGS-treated feeds are significant and showing a clear outperformance of the feed with DDGS-treated. The DDGS-treated can be included to quail feed at a concentration of 20% without negative effects on growth performance.

### Example 5

### De-Watering of Whole Stillage

As mentioned above in the specification, a better de watering capability of the whole stillage results in a wet cake with a higher dry mass. The advantage here is less enemy consumption while drying.

For that example the de watering capability of the whole stillage during the centrifugation was tested and determined. Two different setups were tested. In the first test the beer after the fermentation was not treated with enzymes (DDGS blank), within the second test the beer was treated with the enzyme composition comprising a xylanase and a 1,3-glucanase after the fermentation (DDGS treated).

The trials were performed on a 301 scale. Each test was carried out as duplicate. The total enzyme concentration added was 400 g/t of beer. After the distillation the fermenter was drained off and the whole stillage was centrifuged to obtain thin stillage and the wet cake.

301 Corn to Ethanol fermentation with enzyme application and DDGS production:
pre milled corn<2 mm particle size
10 kg corn were mixed with tap water at 35° C. t to reach a concentration of ˜32% (w/w)
pH range was 5.6-6.0
temperature was increased to 90° C.
7 ml alpha-amylase (Liquizyme from Novozymes) were added
1‰ antifoam was added (30 ml)
incubation for 90 min at 90° C. and 150 rpm
12 ml Glucoamylase (Novozymes Spirizyme Ultra) was added
addition of 300 ppm ((NH4)2SO4) as nitrogen source
direct inoculation with dry yeast
pH adjustment to pH 5.5
fermentation for 62 h at 33° C. and 150 rpm to obtain the beer
The beer is than treated with enzymes for 6 hours at 37′C
after 68 hours ethanol is removed by distillation obtaining a residue called whole stillage
afterwards the whole stillage is centrifuged at 3000 rpm resulting in thick stillage and thin stillage
concentration of the thin stillage to 50% DM by evaporation—The wet cake is mixed with the thin stillage and dried in a drum dryer at ca. 120° C. up to a 90% dry matter.

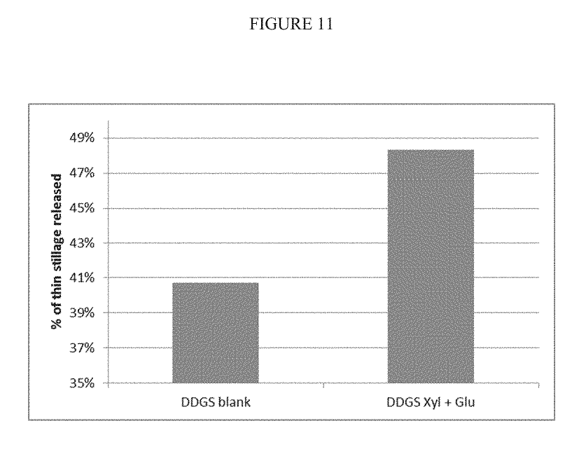

FIG. 11 shows the amount of thin stillage (supernatant) in % of whole stillage removed after centrifugation at 3000 rpm for 10 minutes. Here it was shown that a treatment with the enzyme composition comprising a xylanase and a 1,3-glucanase increases the dewatering capability by 18.7%.

### Example 6

### In-Vitro Digestibility DDGS

Protein release pepsin/HCL digestion and free amino group determination measured by TNBS.

In the following the In vitro protein digestibility assay is described:

DDGS samples are digested by a Pepsin/HCl solution: 0.1 M HCl with 20 mg/ml Pepsin. (Pepsin from porcine gastric mucosa, powder, 400-800 units/mg protein, Sigma: P7125) The supernatant is used for the determination of free amino groups by the TNBS assay.

### Protein Digestion:

From each DDGS sample an amount of 0.5g is taken. Each tests will be done as duplicate
sample is to solubilized with 8 ml Pepsin/HCL solution
Each vial is to shake with a Vortexer and incubate for the next 60 min at 40° C.
After 60 min. the sample is to centrifuge for 20 min at 4000 rpm. The centrifuge temperature is to setup to 4° C.
After the centrifugation the supernatant is to store on ice until the next step regards the free amino group determination

### Protein Determination:

2,4,6-Trinitrobenzene Sulfonic Acid (TNBSA or TNBS (2,4,6-Trinitrobenzene Sulfonic Acid) is a rapid and sensitive assay reagent for the determination of free amino groups. Primary amines, upon reaction with TNBSA, form a highly chromogenic derivative, which can be measured at 335 nm (see figure). Qualitative measurements of amines, sulfhydryls or hydrazides, 3 and quantitative measurements of free-amino groups of L-lysine have also been obtained using TNBSA.

Preparation of buffers

| No | Component |
|---|---|
| 1 | 1% SDS |
| 2 | 0.1M HCl |
| 3 | 0.025% TNBS in 87.5 mM Na-Phosphat pH 8.2 |
| 4 | 100 mM Glycin stock in 1% SDS |

Sampling (in 96-well-plate):
1. For standard curve:
   Glycin start 2500 μM, 2× diluted in 1% SDS

Glycin row

2500
1250
625
312.5
156.25
78.125
39.0625
0

2. Samples, (diluted 1000×) with 1% SDS
   Reaction (96 well PCR plate):
1. 15 μl of Glycin row or samples+90 μl of buffer No 3 (see buffer list)
2. PCR plate is incubated in PCR cycler, program:
   50° C. for 30 min
   4° C. for 10 min
3. Take out from PCR cycler, mix properly

### Measurement:

50 μl of 3+50 μl of buffer No 2 (see buffer list), mix, measure absorption at 340 nm

Two different setups were tested. In the first test the beer after the fermentation was not treated with enzymes (DDGS-blank), within the second test the beer was treated with an enzyme composition comprising a xylanase and a 1,3-glucanase as main activities after the fermentation (DDGS-treated).

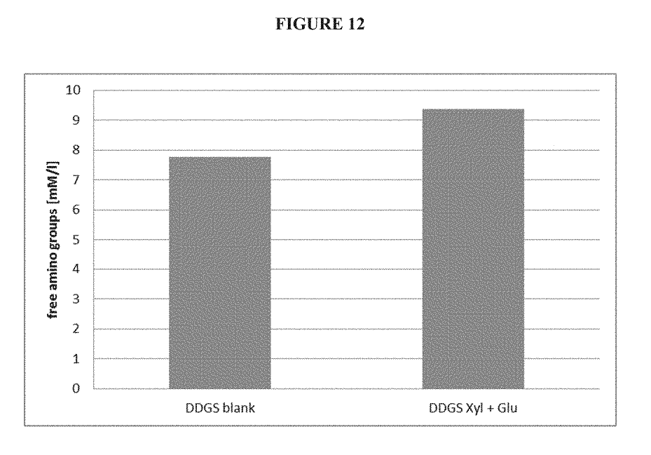

The enzyme treated DDGS was produced with the enzyme composition comprising a xylanase and a 1,3-glucanase as main activities with a total concentration of 400 g/t beer. FIG. 12 is showing the results.

FIG. 12 shows the release of free amino groups with different substrates. The DDGS-treated shows an increased concentration of free amino groups by 20.4%, determined by TNBS assay

Therefore it can be clearly shown that compared to the DDGS-blank the protein digestibility with Pepsin/HCL is increased by DDGS-treated. This results in an improved nutritional quality of the DDGD as a by-product of the fermentation process and therefore in a high quality animal feed.

### Example 7

### Protein Solubilisation in Water

Two different setups were tested. In the first test the beer after the fermentation was not treated with enzymes (DDGS-blank), within the second test the beer was treated with the enzyme composition comprising a xylanase and a 1,3-glucanase after the fermentation (DDGS treated). The enzyme DDGS-treated was produced with the enzyme composition with a total concentration of 400 g/t beer.

For that test 0.5 g DDGS was solubilized with 8 ml distilled water and shacked with a Vortexer. The solution was incubated for 60 min at 40° C. After a centrifugation step (20 min, 4000 rpm, 4° C.) the supernatant was tested with the TNBS assay (see example 6) to determine the release of free amino groups.

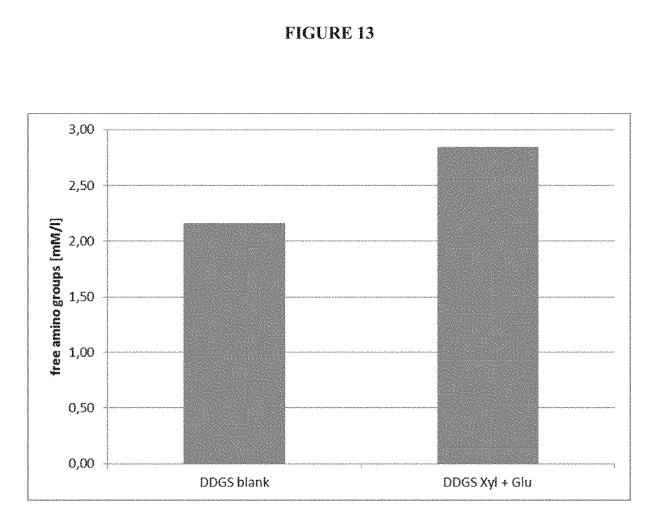

In FIG. 13 the release of free amino groups of DDGS in water is shown. The DDGS-treated is showing an increase of free amino groups by 31.4%. The fiber structure of the DDGS is changed in that way, that the proteins are able to release easier in water solution than untreated DDGS.

This result in an improved nutritional quality of the DDGD as a by-product of the fermentation process and therefore in a high quality animal feed.

### Further Examples

### 1. Material and Methods

1.1 Processes For Producing Fermentation Products (e.g. mash)

In one embodiment, the process of the production of ethanol form corn was performed as follows:

a) Reducing the Particle Size of the Starch-Containing Material by Milling:
corn was milled to <2 mm particle size

b) Forming a Slurry Comprising the Starch-Containing Material and Water
10 kg corn were tap water at 35° C. to obtain a ˜31.25% solid solution, total volume 30 liter
pH range was 5.6-6.0

c) Liquefying of the Starch-Containing Material
temperature was increased to 90° C.
7 ml alpha-amylase (Novozymes Liquozyme SC) were added
1‰ antifoam was added (30 ml)
incubation for 90 min at 90° C. and 150 rpm
slurry was cooled to 30° C., pH adjusted to ˜4 with 1 NH₂SO₄

### d) Saccharifying of the Liquefied Material Obtained

12 ml Glucoamylase (Novozymes Spirizyme Ultra) was added

e) Fermentation
Yeast propagation: 200 ml YNB-starch medium was incubated overnight (30° C., 150 rpm) and inoculated with 2 g yeast (SIHA Amyloferm), the complete pre-culture was added to the fermentation
addition of 300 ppm ((NH4)2SO4) (10 g) as nitrogen source
yeast addition
pH was titrated to 4.0 ammoniac solution (25%), supplies further nitrogen
incubation for max. 48 h at 30° C. and 100 rpm
2-ml samples were taken every 12 hrs to monitor fermentation progress (sugar-, ethanol concentration)
   a. Enzymes

The following enzymes listed in Table 7 were used alone or in different combinations. Enzyme product activity determination is presented in C).

TABLE 7

| Typ | Activity | Shortname |
|---|---|---|
| beta 1-3 Glucanase | 10017 U/g | Glu1 |
| beta 1-3 Glucanase | 763 U/ml | Glu5 |
| beta 1-3 Glucanase | 2588 U/ml | Glu9 |
| beta 1-3 Glucanase | 1469 U/g | Tre6 |
| Xylanase | 1835 U/g | |
| Xylanase | 17047 U/g | BluZyD3 |

b. Enzyme Product Activity Determination:

Substrates: Glucanase: beta-glucan from barley, low viscosity (Megazyme)
Xylanase: Xylan from Birchwood (Sigma)

Substrates were dissolved in buffer to a concentration of 0.8% (w/v)

### Buffer: 50 mM NaAcetat, pH 4.5

The Enzymes were diluted in buffer, the right enzyme concentration must be determined for each enzyme. A 90 μl substrate and 10 μl enzyme solution were mixed. A blank was measured replacing enzyme solution with water. Incubation for 30 min at 37° C. (followed from 10 min at 4° C.). Reducing sugars were measured after mixing of 50 μl of the incubated substrate—enzyme mix with 50 μl DNSA-Reagent.

The activity is calculated as Units per μl or mg of enzyme product. 1 unit is defined as the amount of formed reducing ends in μmol per minute. The enzyme activities are shown in Table 1.

### 1.2 Analysis of Beta-Glucans

Analysis of beta-glucans was carried out with the Yeast beta Glucan Kit K-EBHLG from Megazyme, but one fourth of the described volume in the instructions was used. The analysis was based on the specific enzymatic digestion of (1-3)(1-6)-beta-glucans to glucose. Controls carried out without enzymatic digestion of glucans provide the background concentrations of glucose, which have to be subtracted from the samples.

### 1.3 Enzymatic Digestion of Beta-Glucans

200 μL 1.2 M Na acetate was added to a 50 μL sample, which had been pipetted into an Eppendorf tube. Samples were treated with 5 μL Glucazyme™ an enzyme mixture containing beta-glucanase, beta-glucosidase and chitinase, then stirred by a plastic spatula and subsequently kept for 16 h at 40° C. in an incubator. Glucazyme™ was left out in controls and 5 μL of distilled water was added instead.

1.4 Analysis of Glucose Monomers Derived From Beta-Glucans

25 μL both from the digestion samples and controls was added to 1000 μL GOPOD reagent (containing buffer adjusted to pH 7.4, glucose oxidase, peroxidase and 4-aminoantipyrine p-hydroxybenzoic acid) previously adjusted to 40° C. in a water bath in a photometer cuvette (Rotilabo®-disposable cuvettes, polystirol). The cuvettes were incubated at 40° C. for 30 minutes and the optical density at 510 nm was determined against air in the light path.

1.5 Calculation of Glucose

Calculation of D-glucose concentration of samples and controls was carried with a glucose standard solution (1.5 g glucose/L). The absorbance of the water blank was subtracted from the absorbance of the sample, thereby obtaining ΔA_{D-glucose}_{_}ₛₐₘₚₗₑ. This was also perforated with the glucose standard ΔA_{D-glucose}_{_}_{standard}. Calculation of the glucose concentration of samples and controls was carried out by dividing ΔA_{D-glucose} by ΔA_{D-glucose}_{_}_{standard} *1.5 g/L*dilution factor.

1.6 Calculation of Beta-Glucans

Glucose concentrations of the controls were subtracted from samples treated with Glucazyme™ providing the concentrations of beta-glucans in g/L glucose.

Calculation of □alculation of concentrations of beta-glucans in g/L glucose.amples treated wi

The calculated beta-glucan concentrations (in glucose/L) of the untreated yeast cell and mash samples (i.e. controls) were set to 100%. From the data of beta-glucans in g/L glucose the relative concentrations with reference to the control was calculated.

1.7 Analysis of Manno-Oligosaccharides

Manno-oligosaccharides were analyzed by a varied method presented by Dallies N. et al. 1998 (Dallies N, François J, Paquet V. 1998. François J. Paquet V. A new method for quantitative determination of polysaccharides in the yeast cell wall. Application to the cell wall defective mutants of Saccharomyces cerevisiae. Yeast. 14(14):1297-1306), in which mannose concentrations were analyzed in samples, which had been treated after hot acid hydrolysis and without treatment (controls). The difference amounts of the mannose concentration of samples treated by hot acid hydrolysis and controls give the concentrations of manno-oligosaccharides (in concentration of the mannose oligomer) in the samples.

1.8 Hydrolysis of Manna-Oligosaccharides

500 μl M HCl was added to a 50 μL sample, which has been pipetted into a 1.5 ml Eppendorf tube. For hot acid hydrolysis the sample was kept at 95° C. on an Eppendorf thermomixer shaken at 300 rpm for 2 hours. After heating the tube was cooled in an ice-bath for 5 minutes and 500 μl 2 M NaOH was added into the tube. 50 μl of the sample was used for enzymatic glucose/fructose/mannose analysis. Because determination of mannose was affected by high concentrations of glucose and fructose these sugars were also determined.

Enzymatic analysis of glucose, fructose and mannose

Glucose, fructose and mannose were analyzed in a disposable semi-micro (1.5 mL) photometer cuvette (Rotilabo®-disposable cuvettes, polystirol) at 340 nm at 25° C. against air in the light path of the photometer Cary 50 (Varian) according to the D-mannose. D-fructose and D-glucose assay procedure of the analysis kit K-MANGL by the company Megazyme, but using half of the final volume as stated in the instructions: 1.26 mL instead of 2.52 mL for glucose analysis, 1.27 mL instead of 2.54 mL for fructose and 1.28 mL instead of 2.56 mL, for mannose.

1 mL of distilled water was added to 50 μL of the sample. Then 100 μl of bottle 1 of kit K-MANGL containing triethanolamine buffer pH 9 with magnesium chloride was added followed by 100 μL of bottle 2 containing ATP and NADP. After each step the reaction mixture was stirred by plastic spatula. After 15 minutes incubation at 25° C. optical density was determined at 340 nm against air in the light path giving the background (blank) extinction of the sample (A1). For analysis of glucose 10 μL of a suspension of hexokinase and glucose-6-phosphate dehydrogenase from bottle 3 was pipetted, stirred by a plastic spatula and incubated for 45 Minutes at 25° C. Then the optical density at 340 nm (A2) was determined like A1. For analysis of fructose 10 μL of a suspension of phosphoglucose isomerase from bottle 4 was added, stirred by a plastic spatula and incubated for 30 minutes at 25° C. Then the optical density at 340 nm (A3) was determined like A1. For analysis of mannose 10 μL of a suspension of phosphomannose isomerase from bottle 5 was added, stirred by a plastic spatula and incubated for 45 minutes at 25° C. Then the optical density at 340 nm (A4) was determined like A1.

1.9 Calculation of glucose, fructose and mannose

The absorbance differences (A2−A1) for both blank and sample were determined. The absorbance difference of the blank was subtracted from the absorbance difference of the sample, thereby obtaining ΔA_{D-glucose}.

The absorbance differences (A3−A2) for both blank and sample were determined. The absorbance difference of the blank was subtracted from the absorbance difference of the sample, thereby obtaining ΔA_{D-fructose}.

The absorbance differences (A4−A3) for both blank and sample were determined. The absorbance difference of the blank was subtracted from the absorbance difference of the sample, thereby obtaining ΔA_{D-mannose}.

The concentration of D-glucose, D-fructose and D-mannose can be calculated as follows:

$$c = \frac{V * MW}{ɛ * d * v} * A [ g / L ]$$

where:
V=final volume [mL]; MW=molecular weight of D-glucose, D-fructose or D-mannose [g/mol]
ε=extinction coefficient of NADPH at 340 nm=6300 [1*mol⁻¹*cm-1]; d=light path [cm]
v=sample volume [mL]

It follows for D-glucose:
c=(1.26*180.16*ΔA_{D-glucose})/(6300*1*0.05)[g/L]=0.7206*ΔA_{D-glucose}[g/L]

for D-fructose:
c=(1.27*180.16*ΔA_{D-fructose})/(6300*1*0.05)[g/L]=0.7264*ΔA_{D-fructose}[g/L]

for D-mannose:
c=(1.28*180.16*ΔA_{D-mannose})/(6300*1*0.05)[g/L]=0.7321*ΔA_{D-glucose}[g/L]

If the sample has been diluted during preparation, the result must be multiplied by the dilution factor, F.

Calculation of manno-oligosaccharides

The concentrations of mannose of samples (controls), which were not treated by hot acid hydrolysis, were subtracted from the samples treated by hot acid hydrolysis. The results give the concentrations of manno-oligosaccharides in mannose g/L.

### 2. Example 1

Improvement of Beta-Glucan in DDGS Extracts Derived From Enzyme Treated Mash

For analysis of the effects of enzyme treatment on mash, from which DDGS extracts were generated, 250 ml Erlenmeyer flasks were used as reaction batches, in which 200 ml of fermentatively produced mash were filled in. Enzyme stock solutions (100 mg/mL) solved in 50 mM Na-acetate, pH 4.5 buffer were prepared and 800 μL of the stock solution was added to the Erlenmeyer flask and incubated at 37° C. for 18 h at 150 rpm. Then the mash samples were dried (48 h at 60° C.) to produce DDGS. For the analysis of soluble beta-glucans, 1 g DDGS was dissolved in 3 ml water for 20 min at 50° C. The residual solids were removed by centrifugation at 4000 rpm for 15 minutes at 4° C. and then the beta-glucan concentrations of the supernatants were analyzed according to (d).

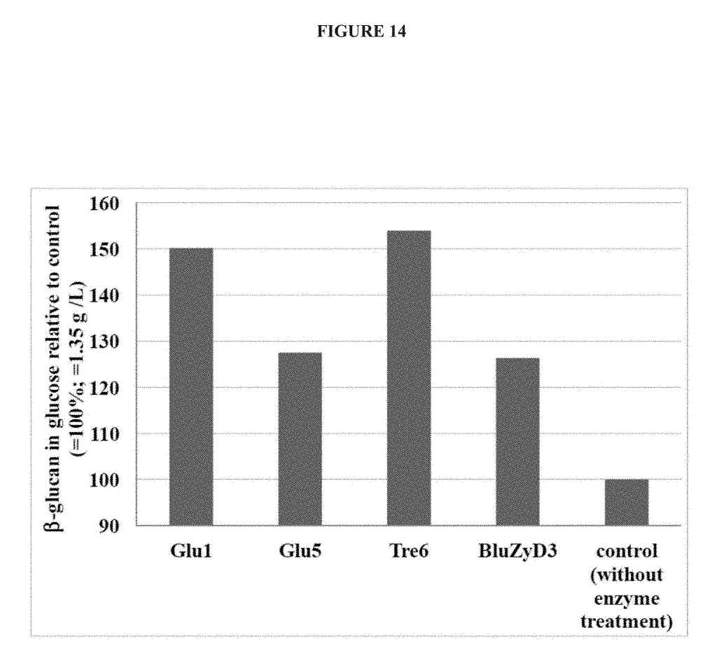

By adding Glu1, Glu9, Tre6 or BluZyD3 to the mash beta-glucan concentration, i.e. beta-glucan content, was increased compared to the control (sample without enzyme treatment) as presented in table 8 and FIG. 14.

TABLE 8

| | beta-glucan | relative |
|---|---|---|
| | in g glucose/L | to control [100%] |
| Glu1 | 2.02 | 150 |
| Glu9 | 1.72 | 127 |
| Tre6 | 2.08 | 153 |
| BluZyD3 | 1.70 | 126 |
| control | 1.35 | 100 |
| (without enzyme treatment) | | |

FIG. 14 shows the release of beta-glucans (in glucose) in DDGS extracts derived from enzymatic hydrolysis of mash treated by different enzymes. By adding Glu1, Glu9, Tre6 or BluZyD3 to the mash beta-glucan concentrations were increased in DDGS extracts compared to the control (without enzyme treatment).

### 3. Example 2

Improvement of Manno-Oligosaccharides in DDGS Extracts Derived From Enzyme Treated Mash

For analysis of the effects of enzyme treatment on mash, from which DDGS extracts were generated, 250 ml Erlenmeyer flasks were used as reaction batches, in which 200 ml of fermentatively produced mash were filled in. Enzyme stock solutions (100 mg/mL) solved in 50 mM Na-acetate, pH 4.5 buffer were prepared and 800 μL of the stock solution was added to the Erlenmeyer flask and incubated at 37° C. for 18 h at 150 rpm. Then the mash samples were dried (48 h at 60° C.) to produce DDGS. For the analysis of soluble manno-oligosaccharides, 1 g DDGS was dissolved in 3 ml water for 20 min at 50° C. The residual solids were removed by centrifugation at 4000 rpm for 15 minutes at 4° C. and then the manno-oligosaccharides concentrations of the supernatants were analyzed according to (e).

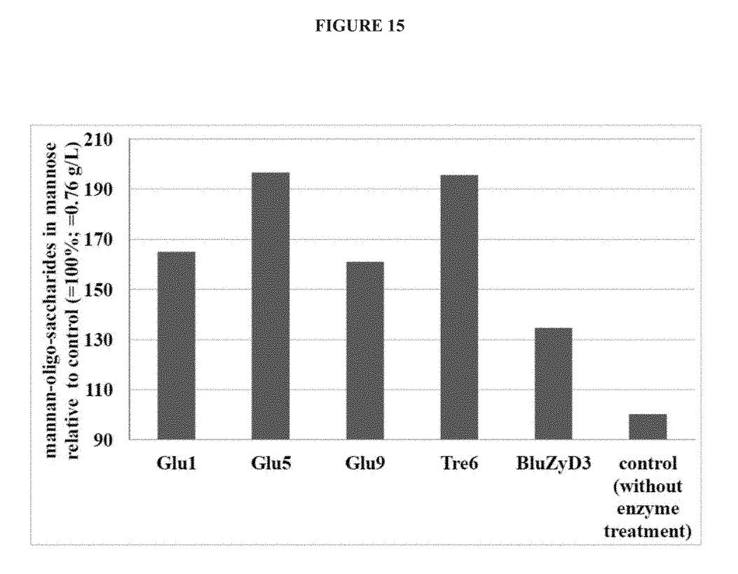

By adding Glu1, Glu5, Glu9, Tre6 or BluZyD3 to the mash manno-oligosaccharide (mos) concentration, i.e. manno-oligosaccharide (mos) content, was increased to the control (sample without enzyme treatment) as presented in table 9 and FIG. 15.

TABLE 9

| | mos in | relative to |
|---|---|---|
| | g mannose/L | control [100%] |
| Glu1 | 1.26 | 165 |
| Glu5 | 1.50 | 197 |
| Glu9 | 1.22 | 161 |
| Tre6 | 1.49 | 196 |
| BluZyD3 | 1.02 | 135 |
| control | 0.76 | 100 |
| (without enzyme treatment) | | |

FIG. 15 shows the release of mannan-oligo-saccharides (in mannose) in DDGS extracts derived front enzymatic hydrolysis of mash treated by different enzymes. By adding Glu1, Glu5, Glu9, Tre6 or BluZyD3 to the mash manno-oligosaccharide (mos) concentrations were increased in DDGS extracts compared to the control (without enzyme treatment).

What is claimed is:

1. A method of recovering oil, comprising (a) converting a starch-containing material into dextrins with an alpha-amylase; (b) saccharifying the dextrins using a carbohydrate source generating enzyme to form a sugar; (c) fermenting the sugar in a fermentation medium into a fermentation product using a fermenting organism, wherein the fermentation medium comprises a xylanase and a pectinase; (d) distilling the fermentation product to form a whole stillage; (e) separating the whole stillage into thin stillage and wet cake; and (f) recovering the oil from the thin stillage.

2. The method according to claim 1, wherein the fermentation product is selected from the group consisting of an acid, an alcohol, and hydrogen.

3. The method according to claim 2, wherein the alcohol is selected from the group consisting of ethanol, butanol, propanol, methanol, propanediol, and butanediol.

1. The method according to claim 2, wherein the acid is selected from the group consisting of lactic acid, propionic acid, acetic acid, succinic acid, malic acid, butyric acid, and formic acid.

5. The method according to claim 1, wherein the starch containing material is obtained from cereals and/or tubers.

6. The method according to claim I, wherein the starch containing material is selected from the group consisting of corn, wheat, barley, rye, millet, sorghum, and milo.

7. The method according to claim 1, wherein the microorganism is selected from the group consisting of a bacteria, a yeast, and a fungi.

8. A process for producing recoverable oil from fermentation processes, wherein the process sequentially comprises the following steps: a) milling whole grain; b) liquefying the gelatinized milled whole grain, in the presence of an alpha-amylase; c) saccharifying the liquefied material in the presence of a glucoamylase; d) fermentation with a microorganism; e) distillation of fermented and saccharified material, thereby providing an ethanol fraction, wherein the liquefied mash is subjected to an effective amount of enzyme activity of a xylanase and a pectinase.

9. The process of claim 8, wherein the microorganism is a bacteria, yeast or fungi.
